(19) United States
(12) Patent Application Publication
(10) Pub. No.: US 2025/0334508 A1
(43) Pub. Date: Oct. 30, 2025

(54) PHOTOTHERMAL EFFECT-BASED MID-INFRARED DETECTING APPARATUS

(71) Applicant: Electronics and Telecommunications Research Institute, Daejeon (KR)

(72) Inventors: Dong Ho SHIN, Daejeon (KR); Eun Sol KO, Daejeon (KR); Hyun Woo SONG, Daejeon (KR); Won Bae CHO, Daejeon (KR)

(73) Assignee: Electronics and Telecommunications Research Institute, Daejeon (KR)

(21) Appl. No.: 19/078,586

(22) Filed: Mar. 13, 2025

(30) Foreign Application Priority Data
Apr. 24, 2024 (KR) 10-2024-0054866

(51) Int. Cl. G01N 21/17; G01N 21/31; G01N 21/3504; G01N 33/497; H04N 25/21; H10F 30/21; H10F 30/22

(52) U.S. Cl. CPC G01N 21/171; G01N 21/314; G01N 21/3504; G01N 33/4972; H04N 25/21; H10F 30/288; G01N 2021/1714; G01N 2021/3155; G01N 2201/0216; G01N 2201/06146; G01N 2201/0696; G01N 2201/121; H10F 30/22

(57) ABSTRACT

The present invention relates to a photothermal effect-based mid-infrared detecting apparatus including an optical sensor that detects visible light, an infrared light layer disposed on the optical sensor and including a material that absorbs a mid-infrared light, and a processor that detects the mid-infrared light by analyzing a sensor signal output from the optical sensor, wherein the optical sensor receives heat generated by the mid-infrared light incident on the infrared light layer and outputs the sensor signal modulated by the heat to the processor.

### CROSS-REFERENCE TO RELATED APPLICATION

This application claims priority to and the benefit of Korean Patent Application No. 10-2024-0054866 filed on Apr. 24, 2024, the disclosure of which is incorporated herein by reference in its entirety.

### BACKGROUND

### 1. Field of the Invention

The present invention relates to an apparatus for detecting a mid-infrared light through a general optical sensor using a photothermal effect.

### 2. Discussion of Related Art

A mid-infrared (MIR) light refers to a light having a wavelength of 2,000 nm to 50,000 nm.

Since absorption spectra of many molecules are concentrated in a wavelength range of the mid-infrared light, the mid-infrared light is very useful for identifying components of the molecules through absorption spectrum analysis.

For this reason, the mid-infrared light is a highly utilized light in many fields such as chemical and biological detection, environmental and hazardous substance monitoring, medicine, telecommunications, astronomy, national defense, and security.

However, since a mid-infrared light optical sensor is more expensive in materials than a silicon sensor and has lower sensitivity (when not amplified) than a silicon sensor has, use of the mid-infrared light optical sensor is limited.

In particular, the use thereof is limited in high sensitivity sensing fields in which light of which power is smaller than pW is detected as in a photo-multiplier tube and an avalanche photodiode that are used for sensing visible light and near-infrared light ranges.

Although research on the mid-infrared light optical sensor that satisfies price and performance is actively being conducted, there are still many improvements in terms of function and performance in a development stage.

The background art of the present invention is disclosed in Korean Patent Registration No. 10-2492134 (registered on Jan. 27, 2023).

### SUMMARY OF THE INVENTION

The present invention is directed to providing a photothermal effect-based mid-infrared detecting apparatus that detects a mid-infrared light at a single photon level based on a photothermal effect by coating an optical sensor with a specific material.

According to an aspect of the present invention, a photothermal effect-based mid-infrared detecting apparatus includes an optical sensor that detects visible light, an infrared light layer disposed on the optical sensor and including a material that absorbs a mid-infrared light, and a processor that detects the mid-infrared light by analyzing a sensor signal output from the optical sensor, wherein the optical sensor receives heat generated by the mid-infrared light incident on the infrared light layer and outputs the sensor signal modulated by the heat to the processor.

The processor may detect the mid-infrared light by analyzing the sensor signal modulated by a photothermal effect of the infrared light layer when the mid-infrared light is radiated while the visible light having a constant intensity is radiated to a surface of the optical sensor.

The processor may calculate a modulation depth of the sensor signal to measure an intensity of a mid-infrared light source.

The processor may correct a fluctuation in the sensor signal based on a homodyne detecting method.

The optical sensor may include any one of a silicon photodiode, an avalanche photodiode, and a silicon photomultiplier.

A detectable wavelength of the infrared light layer may be changed according to a diameter and the number of walls of the material.

The infrared light layer may be formed by coating a surface of the optical sensor with the material that absorbs the mid-infrared light to generate a photothermal effect.

The infrared light layer may be formed of any one of graphene and a carbon nanotube.

According to another aspect of the present invention, a photothermal effect-based mid-infrared detecting apparatus includes a photodiode array that constitutes a spectrometer, an infrared light layer disposed on the photodiode array and including a material that absorbs a mid-infrared light, and a processor that detects the mid-infrared light by analyzing a sensor signal output from the photodiode array, wherein the processor corrects a fluctuation in the sensor signal, analyzes a modulated sensor signal, and detects the mid-infrared light.

The processor may correct a fluctuation in the sensor signal based on a homodyne detecting method.

The processor may radiate visible light having a constant power to the photodiode array and analyze transmission or absorption characteristics of a mid-infrared light range of a sample.

The photothermal effect-based mid-infrared detecting apparatus may further include a mirror that reflects a light wherein the processor may analyze characteristics of light reflected on the mirror.

According to still another aspect of the present invention, a photothermal effect-based mid-infrared detecting apparatus includes an optical sensor that detects a face area of a user, an infrared light layer disposed on the optical sensor and including a material that absorbs a mid-infrared light, a light source that radiates an infrared light toward the user, and a processor that detects the infrared light by analyzing a sensor signal output from the optical sensor, wherein the processor detects alcohol that reacts to the infrared light based on the sensor signal.

The light source may include a first light source that radiates a near-infrared light toward the user and a second light source that radiates the mid-infrared light toward the user.

The processor may detect the alcohol by analyzing the sensor signal according to an absorption wavelength of the alcohol.

The processor may generate an image of distribution of the alcohol exhaled from the user.

The photothermal effect-based mid-infrared detecting apparatus may further include an output unit that outputs an alcohol detection result of the processor, wherein the processor may generate result data related to detection of the alcohol and a concentration of the alcohol and output the result data through the output unit.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a view illustrating a configuration of a photothermal effect-based mid-infrared detecting apparatus according to an embodiment of the present invention;

FIG. 2 is a block diagram schematically illustrating a control configuration of the photothermal effect-based mid-infrared detecting apparatus according to the embodiment of the present invention;

FIGS. 3A and 3B are views illustrating a detection signal of the photothermal effect-based mid-infrared detecting apparatus according to the embodiment of the present invention;

FIGS. 4A and 4B are views illustrating a signal waveform of the photothermal effect-based mid-infrared detecting apparatus according to the embodiment of the present invention;

FIG. 5 is a view illustrating another example of the photothermal effect-based mid-infrared detecting apparatus according to the embodiment of the present invention;

FIG. 6 is a view illustrating still another example of the photothermal effect-based mid-infrared detecting apparatus according to the embodiment of the present invention; and

FIG. 7 is a view illustrating an alcohol detector using the photothermal effect-based mid-infrared detecting apparatus according to the embodiment of the present invention.

### DETAILED DESCRIPTION OF EXEMPLARY EMBODIMENTS

Hereinafter, embodiments of a photothermal effect-based mid-infrared detecting apparatus according to the present invention will be described.

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers. A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

Hereinafter, with reference to the accompanying drawings, embodiments of the present disclosure will be described in detail so that a person skilled in the art can readily carry out the present disclosure. However, the present disclosure may be embodied in many different forms and is not limited to the embodiments described herein.

In the following description of the embodiments of the present disclosure, a detailed description of known functions and configurations incorporated herein will be omitted when it may make the subject matter of the present disclosure rather unclear. Parts not related to the description of the present disclosure in the drawings are omitted, and like parts are denoted by similar reference numerals.

In the present disclosure, components that are distinguished from each other are intended to clearly illustrate each feature. However, it does not necessarily mean that the components are separate. That is, a plurality of components may be integrated into one hardware or software unit, or a single component may be distributed into a plurality of hardware or software units. Thus, unless otherwise noted, such integrated or distributed embodiments are also included within the scope of the present disclosure.

In the present disclosure, components described in the various embodiments are not necessarily essential components, and some may be optional components. Accordingly, embodiments consisting of a subset of the components described in one embodiment are also included within the scope of the present disclosure. In addition, embodiments that include other components in addition to the components described in the various embodiments are also included in the scope of the present disclosure.

Hereinafter, with reference to the accompanying drawings, embodiments of the present disclosure will be described in detail so that a person skilled in the art can readily carry out the present disclosure. However, the present disclosure may be embodied in many different forms and is not limited to the embodiments described herein.

In the following description of the embodiments of the present disclosure, a detailed description of known functions and configurations incorporated herein will be omitted when it may make the subject matter of the present disclosure rather unclear. Parts not related to the description of the present disclosure in the drawings are omitted, and like parts are denoted by similar reference numerals.

In the present disclosure, when a component is referred to as being “linked,” “coupled,” or “connected” to another component, it is understood that not only a direct connection relationship but also an indirect connection relationship through an intermediate component may also be included. In addition, when a component is referred to as “comprising” or “having” another component, it may mean further inclusion of another component not the exclusion thereof, unless explicitly described to the contrary.

In the present disclosure, the terms first, second, etc. are used only for the purpose of distinguishing one component from another, and do not limit the order or importance of components, etc., unless specifically stated otherwise. Thus, within the scope of this disclosure, a first component in one exemplary embodiment may be referred to as a second component in another embodiment, and similarly a second component in one exemplary embodiment may be referred to as a first component.

In the present disclosure, components that are distinguished from each other are intended to clearly illustrate each feature. However, it does not necessarily mean that the components are separate. That is, a plurality of components may be integrated into one hardware or software unit, or a single component may be distributed into a plurality of hardware or software units. Thus, unless otherwise noted, such integrated or distributed embodiments are also included within the scope of the present disclosure.

In the present disclosure, components described in the various embodiments are not necessarily essential components, and some may be optional components. Accordingly, embodiments consisting of a subset of the components described in one embodiment are also included within the scope of the present disclosure. In addition, exemplary embodiments that include other components in addition to the components described in the various embodiments are also included in the scope of the present disclosure.

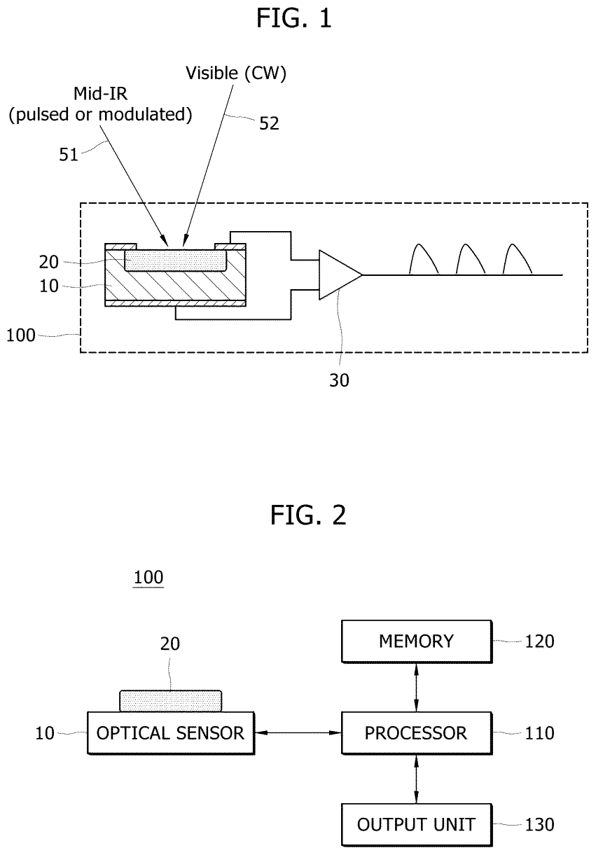

FIG. 1 is a view illustrating a configuration of a photothermal effect-based mid-infrared detecting apparatus according to an embodiment of the present invention.

Referring to FIG. 1, a mid-infrared detecting apparatus 100 according to an embodiment of the present invention may include an optical sensor 10 and a signal processing device 30.

When a visible light (CW) 52 or a mid-infrared light (Mid-IR) 51 is incident, the optical sensor 10 may output a predetermined signal corresponding thereto to the signal processing device 30.

The optical sensor 10 is a general optical sensor that detects the CW 52. The optical sensor 10 may include an infrared light layer 20 of which an upper surface is coated with a material having a photothermal effect.

In general, a phenomenon in which most light energy is converted into heat when a material absorbs a mid-infrared light is called the photothermal effect.

The optical sensor 10 is a typical optical sensor generally used in a visible light range, such as a Si photodiode or a Si photomultiplier, which does not react to a wavelength in a mid-infrared light range and has characteristics of generating an electron-hole pair in the visible light range.

The infrared light layer 20 may generate heat when the Mid-IR 51 is incident thereon. The infrared layer 20 may output a predetermined signal to the signal processing device 30 through the photothermal effect using the generated heat.

The infrared light layer 20 may be formed by stacking a medium that absorbs the Mid-IR 51 but does not generate the electron-hole pair and may be formed by applying the medium around a surface of the sensor. The infrared light layer 20 may be formed of a material that absorbs the Mid-IR 51 to generate heat.

For example, the infrared light layer 20 may be formed of a medium that absorbs a mid-infrared light, such as graphene or a carbon nanotube. It is preferable that a metallic carbon nanotube having excellent conductivity is used as the infrared light layer 20, but in some cases, a carbon nanotube with semiconductive property or a carbon nanotube in which the metallic property and the semiconductive property are mixed at a certain ratio may be used. The infrared light layer 20 may change a sensible wavelength by adjusting a diameter and the number of walls of the carbon nanotube.

The infrared light layer 20 may not directly generate a photocurrent, convert all absorbed photons into heat, and transfer the heat to the optical sensor 10.

In this way, the optical sensor 10 may output a modulated signal due to the photothermal effect caused by the Mid-IR 51 incident on the infrared light layer 20.

When the pulsed or chopped Mid-IR 51 is radiated on the optical sensor 10 while the optical sensor 10 is irradiated with the CW 52 having a certain intensity, the heat from the infrared light layer 20 may be transferred, and an additional current may be generated by the photothermal effect.

The optical sensor 10 may output a sensor signal to the signal processing device 30 in a form in which the photocurrent is modulated over time.

The signal processing device 30 may include at least one of an OP-AMP or a comparator, receive the signal from the optical sensor 10, detect the modulated signal, and output a light source detection result.

FIG. 2 is a block diagram schematically illustrating a control configuration of the photothermal effect-based mid-infrared detecting apparatus according to the embodiment of the present invention.

Referring to FIG. 2, the mid-infrared detecting apparatus 100 may detect the mid-infrared light by receiving the sensor signal from the optical sensor 10 in which the infrared light layer 20 is formed.

The mid-infrared detecting apparatus 100 may include the optical sensor 10 in which the infrared light layer 20 is formed, a memory 120, a processor 110, and an output unit 130. In this case, the memory 120, the processor 110, and the output unit 130 may be included in the signal processing device 30.

The memory 120 may store the sensor signal input from the optical sensor 10, signal data according to a wavelength of a light, and detection result data for the light. Further, the memory 120 may store data and calculation result data for signal comparison and calculation. The memory 120 may include at least one of a signal detecting algorithm, a light source classifying algorithm, and a signal intensity measuring algorithm.

The memory 120 may include a random access memory (RAM), and a nonvolatile memory such as a read only memory (ROM), and an electrically erasable programmable ROM (EEPROM) and storage means such as a flash memory.

The output unit 130 may output a light source detection result for the sensor signal of the optical sensor 10. The output unit 130 may include at least one of a speaker, an operation lamp, and a display.

The output unit 130 may output a predetermined signal waveform according to a determination result of the processor 110. The output unit 130 may output notification for detection of the mid-infrared light in response to the determination result of the processor 110. The output unit 130 may output the notification in the form of at least one of an effect sound, a warning sound, an operation lamp, a notification message, and an image.

In some cases, the mid-infrared detecting apparatus 100 may further include a communication unit. The mid-infrared detecting apparatus 100 may transmit the detection result of the light source to the outside through the communication unit.

The processor 110 may include at least one microprocessor and may be operated based on data stored in the memory 120.

The processor 110 may detect the mid-infrared light based on comparison between the sensor signal output from the optical sensor 10 and a signal stored in the memory 120.

The processor 110 may analyze the sensor signal output from the optical sensor 10, detect the modulated signal, and measure a modulation depth of the modulated signal. The processor 110 may measure an intensity of the mid-infrared light according to the modulation depth.

The processor 110 may generate detection result data for whether the mid-infrared light is detected and the intensity of the light source, store the detection result data in the memory 120, and output notification for the detection result data through the output unit 130.

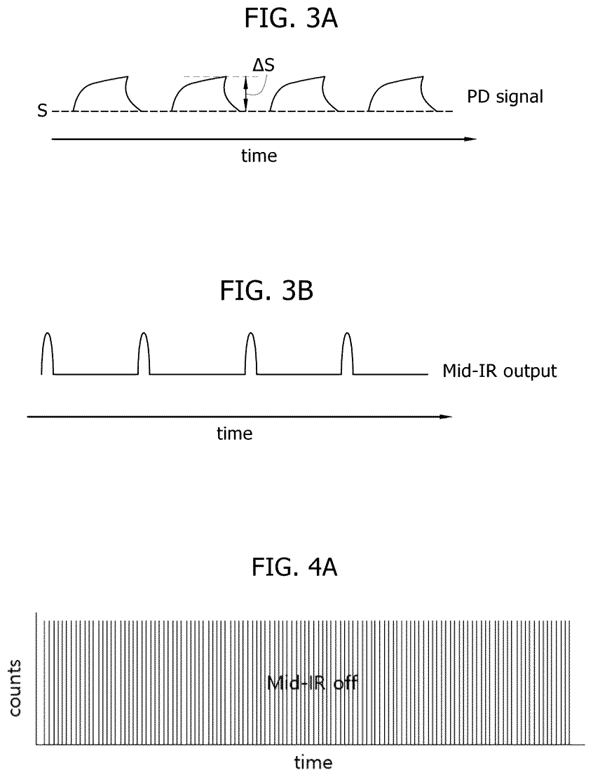

FIGS. 3A and 3B are views illustrating a detection signal of the photothermal effect-based mid-infrared detecting apparatus according to the embodiment of the present invention.

As illustrated in FIG. 3A, in the optical sensor 10, due to a heat conduction mechanism of heat transferred from the infrared light layer 20, when the mid-infrared light having a short pulse width is radiated, a time may be consumed for heating and cooling the sensor, and thus the output sensor signal may be modulated.

The optical sensor 10 has a signal value having a constant level when the mid-infrared light is not radiated, and outputs a modulated signal S when the mid-infrared light is radiated.

The processor 110 may evaluate the intensity of the mid-infrared light by measuring a modulation depth ΔS from the signal S of the optical sensor 10. Here, a magnitude of the modulation depth ΔS varies in proportion to the intensity of the radiated mid-infrared light.

As illustrated in FIG. 3B, the processor 110 may detect the mid-infrared light based on a time point at which the signal is modulated at a predetermined value.

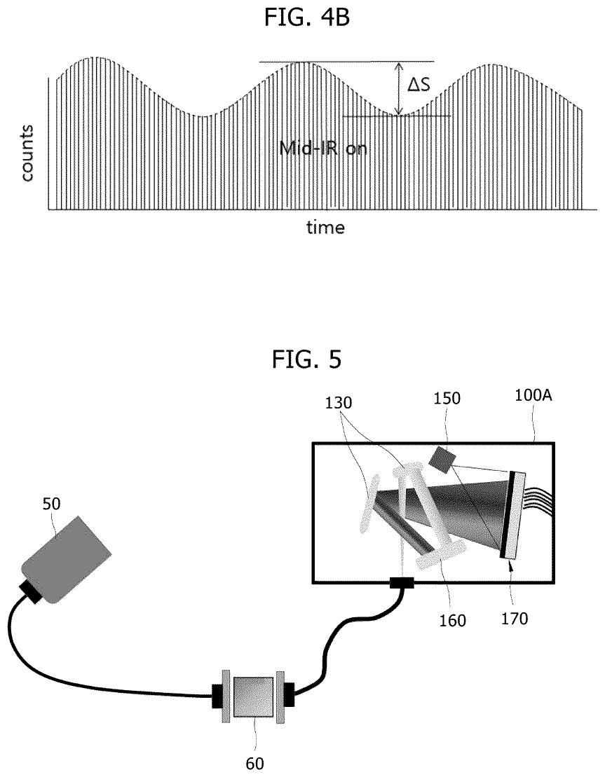

FIGS. 4A and 4B are views illustrating a signal waveform of the photothermal effect-based mid-infrared detecting apparatus according to the embodiment of the present invention.

Referring to FIGS. 4A and 4B, the mid-infrared detecting apparatus 100 may use a time-correlated single-photon counting (TCSPC) technique.

A sensor signal illustrated in FIG. 4A is a TCSPC output value in an ideal case. Small fluctuations may occur in most sensor signals, and thus the sensor signals need to be corrected.

The mid-infrared detecting apparatus 100 may measure light at a single photon level in order to detect a minute change in the mid-infrared light.

The mid-infrared detecting apparatus 100 may detect the mid-infrared light using a detector such as a silicon photomultiplier (SiPM), PMT, or APD in addition to a photodiode (PD) as the optical sensor 10.

The mid-infrared detecting apparatus 100 may detect the mid-infrared light by inputting a signal output from the detector (e.g., PMT or APD) to a signal processing device (e.g., a TCSPC circuit).

When there is no mid-infrared light, the mid-infrared detecting apparatus 100 represents a constant photon counting value as illustrated in FIG. 4A, and when the mid-infrared light source is incident, the counting value is modulated by the photothermal effect.

Accordingly, as illustrated in FIG. 4B, the mid-infrared detecting apparatus 100 may calculate the intensity of the mid-infrared light by calculating the modulation depth ΔS.

The mid-infrared detecting apparatus 100 may precisely measure the mid-infrared light by detecting a minute change in the mid-infrared light using a detector in addition to the photodiode as an optical sensor.

FIG. 5 is a view illustrating another example of the photothermal effect-based mid-infrared detecting apparatus according to the embodiment of the present invention.

Referring to FIG. 5, a mid-infrared detecting apparatus 100A may be applied to a photodiode array (PDA) 170 constituting a spectrometer.

The mid-infrared detecting apparatus 100A may have the infrared light layer 20 formed by applying metallic graphene having absorption properties to the entire mid-infrared light range on the PDA 170.

The mid-infrared detecting apparatus 100A may use a SiPM-based array instead of the PDA 170.

The mid-infrared detecting apparatus 100A may constitute an apparatus that evenly emits visible light (monochromatic light) having a constant power on either the PDA 170 or the SiPM-based array and analyzes characteristics of transmitting or absorbing a signal 50 in the mid-infrared light area of a sample 60 in the spectrometer.

Further, the mid-infrared detecting apparatus 100A may constitute reflective optical systems 150 to 180 including a source 150, a mirror 180, a grating 160, and an array 170 and thus may analyze optical characteristics based on reflective characteristics of the sample.

When the SiPM-based array is used in the mid-infrared detecting apparatus 100A, an array output signal is input to the signal processing device 30 (e.g., a TCSPC array circuit), and the mid-infrared detecting apparatus 100A analyzes a signal in a photon counting method. Here, a light emitting diode (LED) or a laser diode (LD) may be used as a visible light source, and a wavelength may be selected and used in a range of 400 nm to 900 nm depending on experimental conditions.

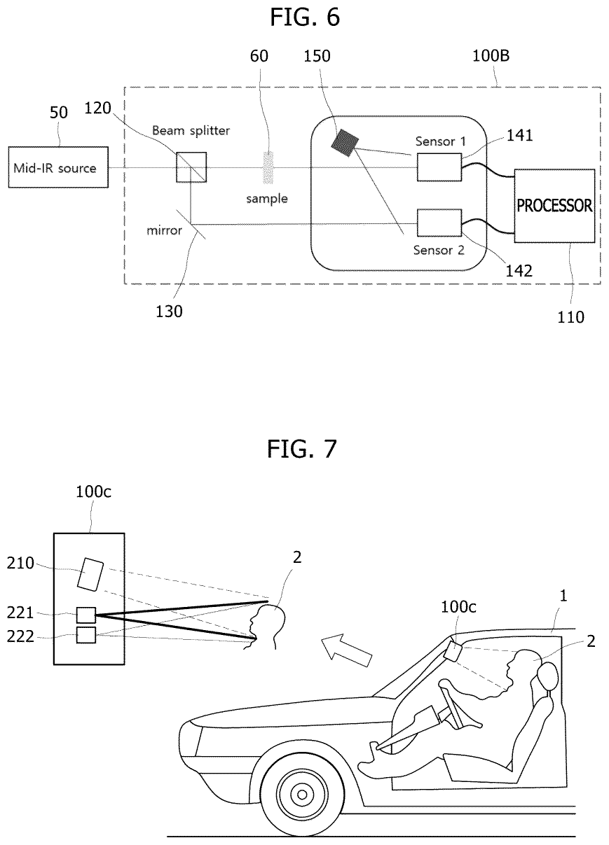

FIG. 6 is a view illustrating still another example of the photothermal effect-based mid-infrared detecting apparatus according to the embodiment of the present invention.

Referring to FIG. 6, a mid-infrared detecting apparatus 100B may use a homodyne detecting method to minimize a phenomenon in which the sensor signal fluctuates.

In the mid-infrared detecting apparatus 100B, a silicon-based PD or the SiPM may be used as the optical sensor 10.

The mid-infrared detecting apparatus 100B may have the infrared light layer 20 formed by applying graphene or carbon nanotubes to a surface of the optical sensor 10. In this case, the mid-infrared detecting apparatus 100B may have the infrared light layer 20 formed by using carbon nanotubes having absorption characteristics of specific wavelength bands to be detected. In addition, the mid-infrared detecting apparatus 100B may have the infrared light layer 20 formed using a material having absorption characteristics in a specific range of the mid-infrared light, such as SiON, SiN, SiC, ZrO, and CaO.

FIG. 7 is a view illustrating an alcohol detector using the photothermal effect-based mid-infrared detecting apparatus according to the embodiment of the present invention.

Referring to FIG. 7, a mid-infrared detecting apparatus 100C may be installed inside a vehicle 1, detect an alcohol component inside the vehicle 1 based on a mid-infrared light, and determine whether a user (driver) 2 is drunk.

Alcohol strongly absorbs light in wavelength bands of the vicinity of 1,550 nm and 9 μm, which are mainly used as an optical communication wavelength band. A general breathalyzer may perform imaging in this area band, but has a high price, and thus is not suitable for mounting on vehicles. A cavity ringdown spectroscopy-type alcohol sensor has a problem in that accuracy is poor as a gas emitted from by the user during breathing and surrounding air are mixed and diluted.

The mid-infrared detecting apparatus 100C may adjust a sensing wavelength such that the mid-infrared light may be measured in the wavelength bands of 1,550 nm and 9 μm and thus effectively measure alcohol exhaled during a breathing process of the user 2 in the vehicle 1.

While a near-infrared light source 221 and a mid-infrared light source 222 illuminate an entire surface of a face of the user (driver) 2, the mid-infrared detecting apparatus 100C may image distribution of the alcohol that remains in the face for a certain period of time or is exhaled, through the optical sensor 10 or 210.

The alcohol may be distributed at a high concentration around the face, and some alcohol may be dispersed through eyes and nose. Thus, the mid-infrared detecting apparatus 100C may measure the alcohol flowing through the eyes even when the user 2 gets on the vehicle while intentionally wearing a mask.

The optical sensor 10 or 210 may include an image sensor.

The optical sensors 10 and 210 may detect the distribution of the alcohol around the face of the user 2.

The processor 110 may generate an image based on the distribution of the alcohol detected through the optical sensor 10 or 210 and output the image through the output unit 130. Accordingly, the mid-infrared detecting apparatus 100C may accurately detect whether the user 2 is drunk.

The mid-infrared detecting apparatus 100C may be installed not only in the vehicle but also in a gate of a work site.

Thus, the photothermal effect-based mid-infrared detecting apparatus according to an aspect of the present invention can easily constitute a mid-infrared light detector with low costs by forming the infrared light layer by coating the optical sensor that detects visible light with a material that absorbs the mid-infrared light and can easily detect the mid-infrared light through the signal modulated using the photothermal effect of the infrared light layer. Further, the photothermal effect-based mid-infrared detecting apparatus according to an aspect of the present invention can effectively detect whether a vehicle driver is drunk by detecting the alcohol using the infrared light.

A photothermal effect-based mid-infrared detecting apparatus according to an aspect of the present invention can simply and inexpensively constitute a mid-infrared detecting apparatus by coating an optical sensor operated in a visible light range with a material that exhibits a photothermal effect.

A photothermal effect-based mid-infrared detecting apparatus according to an aspect of the present invention can easily detect a mid-infrared light at a single photon level and measure an intensity of a light source in a mid-infrared light area

A photothermal effect-based mid-infrared detecting apparatus according to an aspect of the present invention can effectively detect whether a vehicle driver is drunk by detecting alcohol using an infrared light.

What is claimed is:

1. A photothermal effect-based mid-infrared detecting apparatus comprising:
an optical sensor configured to detect visible light;
an infrared light layer disposed on the optical sensor and including a material that absorbs a mid-infrared light; and
a processor configured to detect the mid-infrared light by analyzing a sensor signal output from the optical sensor,
wherein the optical sensor receives heat generated by the mid-infrared light incident on the infrared light layer and outputs the sensor signal modulated by the heat to the processor.

2. The photothermal effect-based mid-infrared detecting apparatus of claim 1, wherein the processor detects the mid-infrared light by analyzing the sensor signal modulated by a photothermal effect of the infrared light layer when the mid-infrared light is radiated while the visible light having a constant intensity is radiated to a surface of the optical sensor.

3. The photothermal effect-based mid-infrared detecting apparatus of claim 1, wherein the processor calculates a modulation depth of the sensor signal to measure an intensity of a mid-infrared light source.

4. The photothermal effect-based mid-infrared detecting apparatus of claim 1, wherein the processor corrects a fluctuation in the sensor signal based on a homodyne detecting method.

5. The photothermal effect-based mid-infrared detecting apparatus of claim 1, wherein the optical sensor includes any one of a silicon photodiode or a silicon photomultiplier.

6. The photothermal effect-based mid-infrared detecting apparatus of claim 1, wherein a detectable wavelength of the infrared light layer is changed according to a diameter and the number of walls of the material.

7. The photothermal effect-based mid-infrared detecting apparatus of claim 1, wherein the infrared light layer is formed by coating a surface of the optical sensor with the material that absorbs the mid-infrared light to generate a photothermal effect.

8. The photothermal effect-based mid-infrared detecting apparatus of claim 7, wherein the infrared light layer is formed of any one of graphene and a carbon nanotube.

9. A photothermal effect-based mid-infrared detecting apparatus comprising:
a photodiode array;
an infrared light layer disposed on the photodiode array and including a material that absorbs a mid-infrared light; and
a processor configured to detect the mid-infrared light by analyzing a sensor signal output from the photodiode array,
wherein the processor corrects a fluctuation in the sensor signal, analyzes a modulated sensor signal, and detects the mid-infrared light.

10. The photothermal effect-based mid-infrared detecting apparatus of claim 9, wherein the processor corrects the fluctuation in the sensor signal based on a homodyne detecting method.

11. The photothermal effect-based mid-infrared detecting apparatus of claim 9, wherein the processor radiates visible light having a constant power to the photodiode array and analyzes transmission or absorption characteristics of a mid-infrared light range of a sample.

12. The photothermal effect-based mid-infrared detecting apparatus of claim 9, further comprising a mirror configured to reflect a light,
wherein the processor analyzes characteristics of light reflected on the mirror.

13. A photothermal effect-based mid-infrared detecting apparatus comprising:
an optical sensor configured to detect a face area of a user;
an infrared light layer disposed on the optical sensor and including a material that absorbs a mid-infrared light;
a light source configured to radiate an infrared light toward the user; and
a processor configured to detect the infrared light by analyzing a sensor signal output from the optical sensor,
wherein the processor detects alcohol that reacts to the infrared light based on the sensor signal.

14. The photothermal effect-based mid-infrared detecting apparatus of claim 13, wherein the light source includes:
a first light source configured to radiate a near-infrared light toward the user; and
a second light source configured to radiate the mid-infrared light toward the user.

15. The photothermal effect-based mid-infrared detecting apparatus of claim 13, wherein the processor detects the alcohol by analyzing the sensor signal according to an absorption wavelength of the alcohol.

16. The photothermal effect-based mid-infrared detecting apparatus of claim 15, wherein the processor generates an image of distribution of the alcohol exhaled from the user.

17. The photothermal effect-based mid-infrared detecting apparatus of claim 13, further comprising an output unit configured to output an alcohol detection result of the processor,
wherein the processor generates result data related to detection of the alcohol and a concentration of the alcohol and outputs the result data through the output unit.
